(19) United States
(12) Patent Application Publication
(10) Pub. No.: US 2025/0064308 A1
(43) Pub. Date: Feb. 27, 2025

(54) CAPSULE ENDOSCOPE

(71) Applicant: Ankon Medical Technologies(Shanghai) Co., LTD, Shanghai (CN)

(72) Inventors: Xiaodong DUAN, Plano, TX (US); Shaobang ZHANG, Shanghai (CN)

(73) Assignees: Ankon Medical Technologies(Shanghai) Co., LTD, Shanghai, CN (CN); ANX IP HOLDING PTE. LTD., SG, SG (SG)

(21) Appl. No.: 18/726,099

(22) PCT Filed: Dec. 29, 2022
(86) PCT No.: PCT/CN2022/143642

§ 371 Date: Jul. 1, 2024

(30) Foreign Application Priority Data
Dec. 31, 2021 (CN) 202111673340.7

(51) Int. Cl. A61B 1/04; A61B 1/00; A61B 1/12

(52) U.S. Cl. CPC A61B 1/041; A61B 1/00032; A61B 1/128

(57) ABSTRACT

The present invention provides a capsule endoscope, including: an enclosure; a battery component disposed in an accommodating space enclosed by the enclosure; a circuit control component electrically connected to the battery component and disposed adjacent to the battery component; an image acquisition component electrically connected to the circuit control component; a first thermally conductive element disposed between the battery component and the circuit control component; and a second thermally conductive element in contact with the image acquisition component and the battery component, respectively. The capsule endoscope can timely and effectively transfer heat generated by the image acquisition component and the circuit control component to the battery component, so that the battery component can reach a higher thermal equilibrium temperature, thereby extending working time of the battery component, solving the issue of insufficient battery power, and without significantly increasing structural complexity of the capsule endoscope.

### FIELD OF INVENTION

The present invention relates to the field of micro medical robots, and more particularly to a capsule endoscope.

### BACKGROUND

Wireless capsule endoscope can not only capture images of the esophagus, stomach, and colon and transmit images to the outside of the body, but it can also detect areas such as the small intestine that traditional endoscopes cannot reach. Therefore, capsule endoscope has significant advantages in gastrointestinal tract examination and has received widespread attention.

However, the battery life of the capsule endoscope in working condition is a key factor limiting its ability to perform a complete examination of the gastrointestinal tract. Battery performance has a significant impact on image quality, image capture frequency, and image transmission. A complete gastrointestinal tract examination requires the battery to provide no less than 12 hours of power. Due to the physical size limitations of the capsule endoscope, it is difficult for the silver oxide or lithium battery used inside the capsule endoscope to provide the necessary power for a complete gastrointestinal examination in one go, especially if a stronger light source is used to capture higher resolution images, which requires even more battery power.

With the trend of miniaturization of capsule endoscopes, the batteries typically used in capsule endoscopes are button cells, which have limited circuit capacity.

To ensure that the battery has sufficient power, prior art uses wireless charging to remotely charge the battery in the capsule endoscope. However, this method significantly complicates the structure of the capsule endoscope and increases cost.

Therefore, extending the battery life to capture more images without significantly increasing the structural complexity of the capsule endoscope has become a pressing technical problem that needs to be solved.

### SUMMARY OF THE INVENTION

In order to technically solve above problems of the prior art, the present invention provides a capsule endoscope. The capsule endoscope comprises: an enclosure; a battery component disposed in an accommodating space enclosed by the enclosure; a circuit control component electrically connected to the battery component and disposed adjacent to the battery component; an image acquisition component electrically connected to the circuit control component; a first thermally conductive element disposed between the battery component and the circuit control component; and a second thermally conductive element in contact with the image acquisition component and the battery component, respectively.

The first thermally conductive element comprises an insulating and thermally conductive material layer.

The second thermally conductive element comprises a thermally conductive plate, the thermally conductive plate having a first end in contact with the image acquisition component, a second end in contact with the battery component, and an intermediate end in contact with the circuit control component, or the thermally conductive plate having a first end in contact with the image acquisition component and a second end in contact with the battery component.

The second thermally conductive element comprises a thermally conductive frame and a heat dissipation cavity defined by the thermally conductive frame, and the heat dissipation cavity is filled with a solid-state phase change material.

At the first end where the second thermally conductive element contacts the image acquisition component, the heat dissipation cavity has a roughened inner surface.

The second thermally conductive element comprises a thermally conductive frame and a heat dissipation cavity defined by the thermally conductive frame, the heat dissipation cavity is filled with gas-liquid phase change material for heat dissipation, and the inner wall surface of the heat dissipation cavity has a capillary structure.

At the first end where the second thermally conductive element contacts the image acquisition component, the heat dissipation cavity has an evaporation-promoting structure.

As previously described, the capsule endoscope further comprises a first thermal insulation material layer disposed between the battery component and the enclosure.

As previously described, the capsule endoscope further comprises a second thermal insulation material layer, the image acquisition component is arranged in a space corresponding to end portion of the enclosure, the second thermal insulation material layer is disposed between the image acquisition component and the enclosure, and the second thermal insulation material layer is a transparent thermal insulation material layer.

The second thermal insulation material layer is arranged on the inner wall at the end portion of the enclosure, and the transparent thermal insulation material layer comprises metal organic ester aerogel.

According to the present invention, the capsule endoscope can timely and effectively transfer the heat generated by the image acquisition component and the circuit control component to the battery component, so that the battery can reach a higher thermal equilibrium temperature, thereby extending the working time of the battery, solving the issue of insufficient battery power, and without significantly increasing the structural complexity of the capsule endoscope.

### BRIEF DESCRIPTION OF THE DRAWINGS

The accompanying drawings, which are included to provide a further understanding of the present invention and are incorporated in and constitute a part of the description, illustrate the embodiment(s) of the present invention and together with the description serve to explain the principle of the invention, but do not constitute a limitation of the present invention. In the drawings:

FIG. 1 is a structural schematic view of a capsule endoscope based on the prior art.

FIG. 2 is a structural schematic view of a capsule endoscope according to an embodiment of the present invention.

FIG. 3 is a structural schematic view of a capsule endoscope according to an embodiment of the present invention.

FIG. 4 is a structural schematic view of a second thermally conductive element of the capsule endoscope according to an embodiment of the present invention.

FIG. 5 is a structural schematic view of a second thermally conductive element of the capsule endoscope according to an embodiment of the present invention.

FIG. 6 is a structural schematic view of a second thermally conductive element of the capsule endoscope according to an embodiment of the present invention.

FIG. 7 is a structural schematic view of a second thermally conductive element of the capsule endoscope according to an embodiment of the present invention.

FIG. 8 is a structural schematic view of a capsule endoscope according to an embodiment of the present invention.

FIG. 9 is a structural schematic view of a capsule endoscope according to an embodiment of the present invention.

### DETAILED DESCRIPTION

To more clearly understand the objects, features, and advantages of the present invention, a detailed description is provided below in conjunction with the accompanying drawings and specific embodiments. It should be noted that, where there is no conflict, the embodiments and features described in the present invention can be combined with each other.

Many specific details are set forth in the following description to provide a thorough understanding of the present invention. However, the present invention can be practiced in other ways than those specifically described herein. Therefore, the scope of the present invention is not limited by the specific embodiments disclosed below.

As shown in FIG. 1, the capsule endoscope in the prior art comprises an enclosure 1′, a battery component 2′ disposed in the enclosure 1′, a magnet 7′, a circuit control component 3′, and an image acquisition component 4′. The magnet 7′ is disposed between the circuit control component 3′ and the battery component 2′.

The circuit control component 3′ comprises an application specific integrated circuit (abbreviated as ASIC). The image acquisition component 4′ comprises an illumination unit, a camera, and an image processer, each with a control circuit, and these circuits generate heat during working. The illumination unit can be light emitting diodes. Since the magnet 7′ is disposed between the circuit control component 3′ and the battery component 2′, the heat generated by these circuits during working cannot be promptly transferred to the battery component, but is dissipated to the enclosure of the capsule endoscope and emitted to the outside through the capsule enclosure.

Additionally, the battery component needs to operate in a high-temperature environment because the battery component in the capsule endoscope is usually one or more silver oxide batteries. The temperature has a significant impact on the operating duration of the capsule endoscope because it accelerates the chemical reactions inside the one or more silver oxide batteries, ensuring smooth electron migration.

However, since the enclosure of the capsule endoscope is primarily made of biocompatible polymer materials, which do not generate heat themselves, the heat generated by the circuit operation is transferred through air convection to the cooler enclosure and dissipates outward. This causes the heat to be scattered and not be provided to the battery component.

Therefore, how to make the heat generated by the circuits available for the battery component without increasing the complexity of the structure of the capsule endoscope has become a problem that needs to be solved.

To address this, an embodiment of the present invention provides a capsule endoscope that can timely and effectively transfer the heat generated during the operation of the circuit control component and the image acquisition component to the battery component, ensuring the temperature of the battery component, extending the working time of the battery, without increasing the structural complexity of the capsule endoscope.

### (1) Embodiment 1

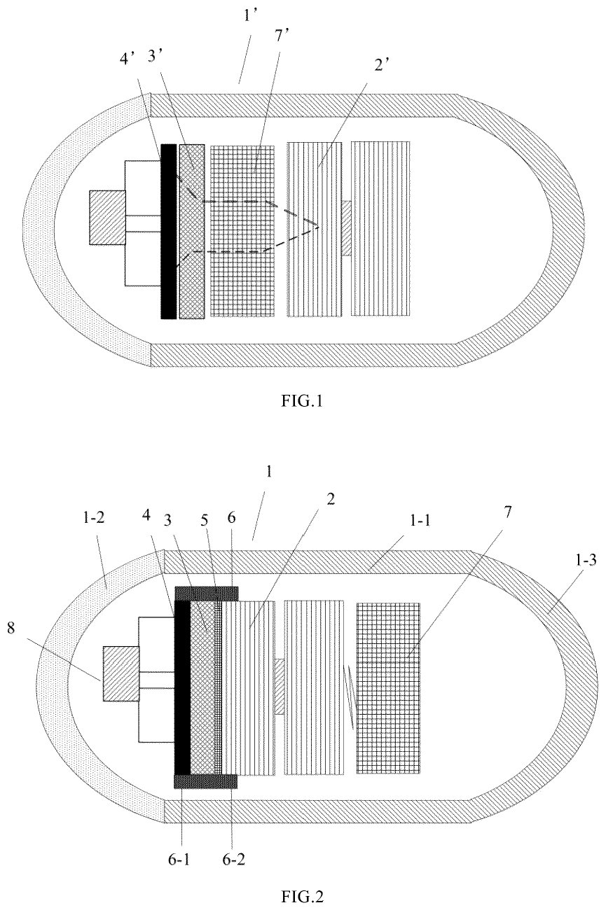

As shown in FIG. 2, this embodiment provides a capsule endoscope, comprising: an enclosure 1; a battery component 2 disposed in an accommodating space enclosed by the enclosure; a circuit control component 3 electrically connected to the battery component 2 and disposed adjacent to the battery component 2; an image acquisition component 4 electrically connected to the circuit control component 3; a first thermally conductive element 5 disposed between the battery component and the circuit control component; and a second thermally conductive element 6 in contact with the image acquisition component and the battery component, respectively.

The enclosure of the capsule endoscope comprises a middle portion 1-1, an end portion 1-2, and an end portion 1-3. The image acquisition component 4 is correspondingly arranged at the end portion 1-2, and a radio frequency component (not shown) is correspondingly arranged at the end portion 1-3. The radio frequency component is used for transmitting images captured by the image acquisition component 4 to outside of the body, and comprises a radio frequency circuit and an antenna.

In the capsule endoscope proposed in this embodiment, the circuit control component 3 is adjacent to the battery component 2, and the magnet 7 is disposed adjacent to the end portion 1-3 of the enclosure 1 of the capsule endoscope. The magnet 7 is not disposed between the circuit control component 3 and the battery component 2. The heat generated when the circuit control component 3 is working can be transferred to the battery component 2, increasing the temperature when the battery component is working.

In the circuit structure of the capsule endoscope, due to the presence of ASIC in the circuit control component, there are various traces and components on the circuit board, which cannot be in close contact with the battery component. There is an air gap between them, resulting in slow transfer of heat to the battery component, and some of the heat will dissipate.

To further increase the efficiency of transferring heat to the battery component, the first thermally conductive element 5 is disposed between the battery component and the circuit control component. The first thermally conductive element 5 can be in direct contact with the battery component and the circuit control component respectively, timely and effectively transferring the heat generated during the operation of the circuit control component to the battery component.

The first thermally conductive element 5 comprises an insulating and thermally conductive material layer. The first thermally conductive element 5 may be an insulating material with good thermal conductivity filled between the battery component 2 and the circuit control component 3. A through hole may also be set in the thermally conductive element 5 to allow wiring for electrical connection between the circuit control component 3 and the battery component 2 to pass through (not shown in the figures).

The material of the first thermally conductive element 5 may comprise or consist of one or more of thermal conductive silicone grease, thermal conductive insulating rubber, thermal conductive potting adhesive, and single-component thermal conductive adhesive. These materials have high dielectric strength, good thermal conductivity, high chemical resistance, and can improve thermal conductivity while avoiding circuit short circuits.

The image acquisition component 4 comprises an illumination unit, a camera, and an image processer, each with a control circuit. It generates heat during operation. The illumination unit can be light emitting diodes. Due to the presence of the circuit control component 3 between the image acquisition component 4 and the battery component 2, the heat generated by the image acquisition component 4 during operation cannot be timely and effectively transferred to the battery component.

The capsule endoscope in this embodiment further comprises a second thermally conductive element 6, which is in contact with the image acquisition component 4 and the battery component 2, forming a thermal pathway between the image acquisition component 4 and the battery component 2 to transfer the heat generated by the image acquisition component 4 to the battery component 2 in a timely manner.

The second thermally conductive element 6 may be a thermally conductive plate, comprising a thermally conductive metal plate or a thermally conductive insulating material. While the metal plate has good thermal conductivity, it may potentially cause a circuit short. In the case where the second thermally conductive element 6 is a metal plate, the part of the second thermally conductive element 6 that contacts the image acquisition component can be insulated using a thermally conductive insulating material. Alternatively, a second thermally conductive element 6 may be formed directly using a thermally conductive insulating material, such as thermally conductive silicone grease, thermally conductive insulation rubber, aluminum nitride, beryllium nitride, aluminum oxide, silicon nitride, etc.

As shown in FIG. 2, the second thermally conductive element 6 has a first end 6-1 in contact with the image acquisition component 4 and a second end 6-2 in contact with the battery component 2. The heat generated during the operation of the image acquisition component 4 is transferred from the first end 6-1 to the second end 6-2, and then transferred to the battery component 2.

The second thermally conductive element 6 shown in FIG. 2 also contacts the circuit control component 3. The middle end of the second thermally conductive element 6 contacts the circuit control component 3, allowing the heat generated by the circuit control component 3 to be transferred to the battery component 2 through the second thermally conductive element 6. It is possible to prevent the heat generated by the circuit control component 3 from dissipating from the side, and further transfer more of the heat generated by circuit control component 3 to the battery component 2.

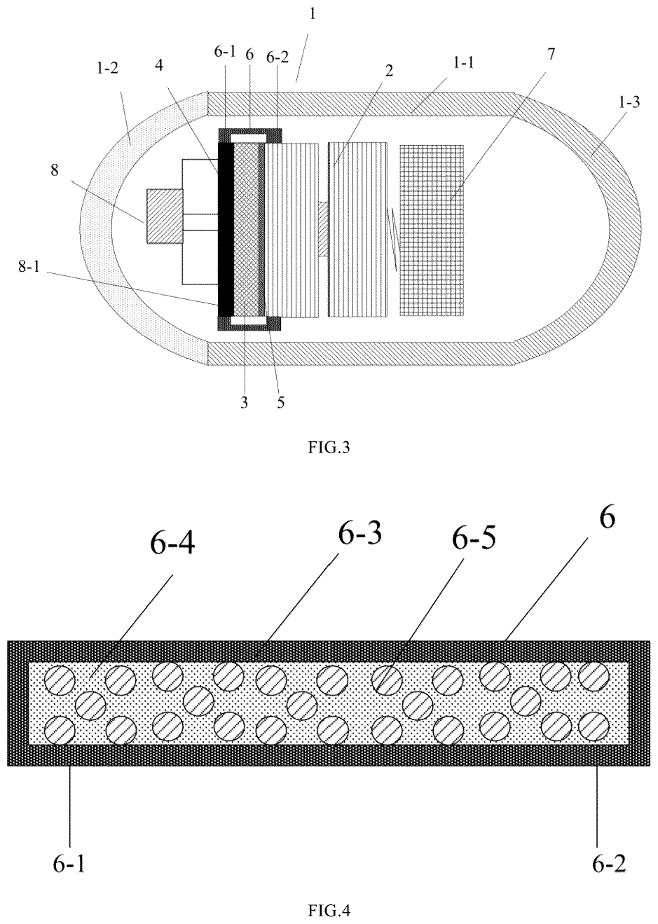

In another embodiment, as shown in FIG. 3, for the convenience of wiring, the second thermally conductive element 6 may not be in contact with the circuit control component 3. Only the first end 6-1 of the second thermally conductive element 6 is in contact with the image acquisition component 4, and the second end 6-2 of the second thermally conductive element 6 is in contact with the battery component 2.

In the capsule endoscope provided in this embodiment, the circuit control component is disposed adjacent to the battery component, thermally conductive elements are disposed between the circuit control component and the battery component, and the thermally conductive elements are respectively arranged in contact with the image acquisition component and the battery component, forming a heat transfer path between the image acquisition component and the battery component, which can effectively transfer the heat generated by the image acquisition component and the circuit control component to the battery component in a timely manner, enabling the battery to reach a higher thermal equilibrium temperature, extending the working time of the battery, solving the problem of insufficient battery power, without excessively increasing the structural complexity of the capsule endoscope.

### (2) Embodiment 2

This embodiment provides a capsule endoscope, comprising: an enclosure 1; a battery component 2 disposed in an accommodating space enclosed by the enclosure; a circuit control component 3 electrically connected to the battery component 2 and disposed adjacent to the battery component 2; an image acquisition component 4 electrically connected to the circuit control component 3; a first thermally conductive element 5 disposed between the battery component and the circuit control component; and a second thermally conductive element 6 in contact with the image acquisition component and the battery component, respectively.

The other structures and materials of the capsule endoscope provided in this embodiment are the same as in Embodiment 1. The difference is that, in order to more effectively transfer the heat generated by the image acquisition component 4 to the battery component 2, in the capsule endoscope of this embodiment, the second thermally conductive element 6 has a hollow structure. As shown in FIG. 4, the second thermally conductive element 6 comprises a thermally conductive frame 6-3 and a heat dissipation cavity 6-4 defined by the thermally conductive frame, and the heat dissipation cavity 6-4 is filled with solid-state phase change material 6-5.

The solid-state phase change material absorbs heat to transition into liquid form, and release heat to the surrounding environment to transition back into solid form from liquid. This process allows for multiple cycles of phase change between solid and liquid, enabling the rapid dissipation of heat generated by a heat source to the cold end in a short amount of time. Furthermore, this phase change cycle does not require external power (electric power or any other energy) other than phase change material, has a simple structure, and low cost.

The first end 6-1 of the second thermally conductive element 6 is in contact with the heat source, which is the image acquisition component 4. The heat generated when the image acquisition component 4 is working is transferred to the solid-state phase change material 6-5. After absorbing heat, the solid-state phase change material turns into liquid and flows to the second end 6-2 of the second thermally conductive element 6. At the second end 6-2, the heat is transferred to the battery component 2 before changing back to solid, and then goes through another cycle after receiving heat again.

The solid-state phase change material 6-5 can quickly transfer the heat generated during the operation of the image acquisition component 4 to the battery component 3, which improves the efficiency of heat transfer, reduces the release of heat from the image acquisition component 4 to the surrounding environment, and enhances the utilization of heat by the battery component.

The solid-state phase change material 6-5 may comprise paraffin, fatty acids, sodium acetate hydrate, and/or sodium sulfate hydrate. The solid-state phase change material 6-5 may also be added with additives such as carbon black, aluminum nitride, and/or aluminum oxide to increase thermal transfer efficiency.

In another embodiment, as shown in FIG. 5, at the first end 6-1 of the second thermally conductive element 6 in contact with the image acquisition component 4, which is the receiving end of heat, the inner surface of the hollow structure is a roughened inner surface with multiple protrusions 6-6, which can increase the contact area of the second thermally conductive element 6 at the heat source end, that is, the contact area with the image acquisition component 4, promote better dissipation of heat to the heat dissipation cavity 6-4, and more quickly transfer heat to the solid-state phase change material 6-5, thereby improving the efficiency of heat transfer from the image acquisition component 4 to the battery component 2.

Multiple protrusions 6-6 can be formed by forming fine particles on the inner surface of the heat dissipation cavity 6-4. The present invention is not limited to this, and other methods may also be used to increase the internal surface roughness of the hollow structure, thereby increasing the contact area between the heat receiving end and the solid-state phase change material. For example, grooves are formed on the inner surface of the hollow structure, a porous structure is set up to form vertical thermally conductive columns, or heat dissipation fins are formed inside the hollow structure, etc. The present invention is not limited to this.

For the second thermally conductive element 6, a metal material with high heat dissipation performance can be used, or a polymer material can be used to form a thermally conductive frame 6-3. The thermally conductive frame 6-3 may be formed by polyethylene with ultra-high molecular weight, polyethylene with ultra-low molecular weight, cross-linked polyethylene, or other polyethylene-based materials with certain mechanical properties.

The second thermally conductive element 6 may be in contact with the battery component, the image acquisition component, and the circuit control component as shown in FIG. 2, or it may only be in contact with the battery component and image acquisition component as shown in FIG. 3.

In the capsule endoscope provided in this embodiment, a thermally conductive element in contact with the image acquisition component and battery component is configured as a structure filled with solid-state phase change material, which can instantly absorb the heat generated during the operation of the image acquisition component and transfer the heat to the battery component, thereby improving the efficiency of heat transfer to the battery component and increasing the utilization of the heat generated by the image acquisition component by the battery component.

### (3) Embodiment 3

This embodiment provides a capsule endoscope, comprising: an enclosure 1; a battery component 2 disposed in an accommodating space enclosed by the enclosure; a circuit control component 3 electrically connected to the battery component 2 and disposed adjacent to the battery component 2; an image acquisition component 4 electrically connected to the circuit control component 3; a first thermally conductive element 5 disposed between the battery component and the circuit control component; and a second thermally conductive element 6 in contact with the image acquisition component and the battery component, respectively.

In this embodiment, the other structures and materials of the capsule endoscope provided are the same as in the Embodiment 1. The difference lies in that, in the capsule endoscope provided in this embodiment, in order to more effectively transfer the heat generated by the image acquisition component 4 to the battery component 2, the second thermally conductive element 6 with a hollow structure is disposed. As shown in FIG. 4, the second thermally conductive element 6 comprises a thermally conductive frame 6-3 and a heat dissipation cavity 6-4 defined by the thermally conductive frame, and the heat dissipation cavity 6-4 is filled with a gas-liquid phase change material. The inner wall surface of the heat dissipation cavity 6-4 has a capillary structure.

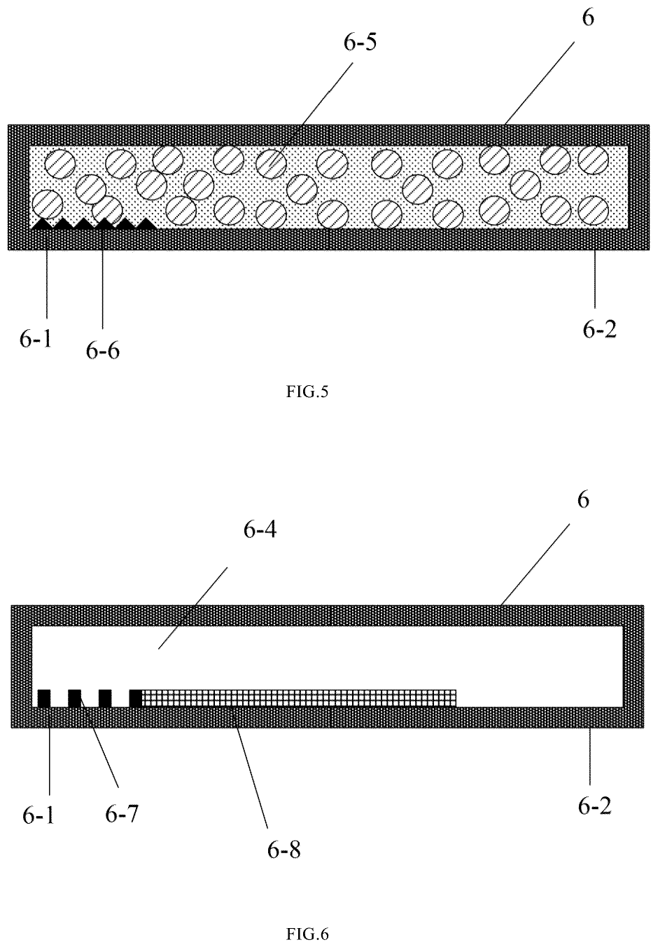

As shown in FIG. 6, the capillary structure on the inner wall surface of the heat dissipation cavity 6-4 is a porous grid structure 6-8. As shown in FIG. 7, the capillary structure on the inner wall surface of the heat dissipation cavity 6-4 is a multi-groove structure 6-9 formed on the inner surface of the heat dissipation cavity. The capillary structure on the inner wall surface of the heat dissipation cavity 6-4 may also be in other forms, and the present invention is not limited to this.

In addition, FIGS. 6 and 7 only schematically show the situation of capillary structures located on the inner wall of the bottom surface of the heat dissipation cavity 6-4. Capillary structures may also be located on the inner wall of the top surface of the heat dissipation cavity 6-4. The present invention is not limited to this.

The gas-liquid phase change material at the heat receiving end, that is, the first end 6-1 of the second thermally conductive element 6 in contact with the image acquisition component 4, is in a liquid form, absorbing heat and evaporating into gas. The gas diffuses to the cold end, that is, the second end 6-2 of the second thermally conductive element 6 in contact with the battery component 2, transferring heat to the battery component 2 and condensing back into gas. The condensed gas returns to the hot end through the capillary action of the capillary structure on the inner wall surface of the heat dissipation cavity 6-4, and starts the next cycle after receiving heat again. It can dissipate the heat generated by the heat source to the cold end in a very short time without the need for external power (electric power or any other energy) other than phase change material to drive the phase change cycle. The structure is simple and cost-effective.

The gas-liquid phase change material may comprise or consist of one or more of alcohol, ammonia, water, acetone, and hydrofluorocarbon refrigerant.

To improve the efficiency of heat transfer, at the first end 6-1 where the second thermally conductive element contacts the image acquisition component, the inner surface of the heat dissipation cavity 6-4 has an evaporation-promoting structure 6-7, which enhances the efficiency of liquid evaporation into gas after receiving heat, thus improving the efficiency of heat transfer.

As shown in FIGS. 6-7, the evaporation-promoting structure 6-7 disposed on the inner surface of the heat dissipation cavity 6-4 is a plurality of thermally conductive columns, which can quickly transfer the heat received from the image acquisition component 4 to the gas-liquid phase change material, promoting the evaporation boiling capability. The evaporation-promoting structure 6-7 may also be configured in other forms, such as evaporative fin, protruding structure, groove channel, etc. The present invention is not limited to this.

In the capsule endoscope provided in this embodiment, a thermally conductive element in contact with the image acquisition component and the battery component is configured as a structure filled with a gas-liquid phase change material, which can instantly absorb the heat generated during the operation of the image acquisition component, transfer the heat to the battery component, and utilize capillary gravity to achieve the flow of the phase change material, improving the efficiency of heat transfer to the battery component and increasing the utilization of the heat generated by the image acquisition component by the battery component.

### (4) Embodiment 4

This embodiment provides a capsule endoscope. The other structures of the capsule endoscope in this embodiment are the same as any of the capsule endoscope structures in Embodiments 1-3, except that in order to further concentrate heat within the space enclosed by the enclosure of the capsule endoscope, which is beneficial for increasing the operating temperature of the battery component, an thermal insulation material layer is provided between the battery component 2 and the enclosure of the capsule endoscope of this embodiment.

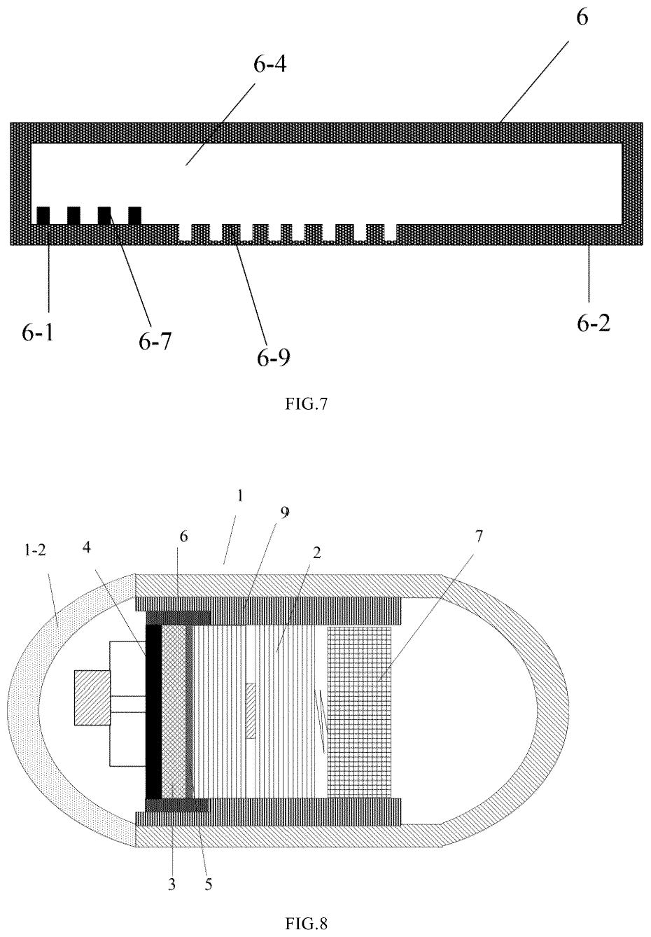

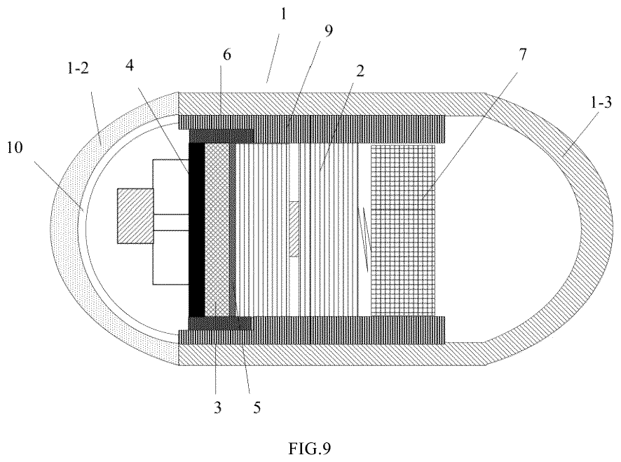

As shown in FIG. 8, a first thermal insulation material layer 9 is provided between the battery component 2 and the enclosure 1. The material of the first thermal insulation layer 9 may be insulating adhesive, aerogel, etc. with poor thermal conductivity, or a solid insulating material with poor thermal conductivity. For example, the first thermal insulation layer may comprise thermal insulation glue in a gel state, such as nanostructured aerogel, or may comprise solid glass fibers, mineral wool insulation fibers, calcium silicate, alumina fibers, cellulose thermal insulation element, polystyrene thermal insulation element, and/or polyurethane foam thermal insulation element.

The first thermal insulation material layer 9 can effectively reduce the thermal convection between the battery component and the enclosure, better concentrate the heat inside the enclosure of the capsule endoscope, allowing the battery to reach a higher thermal equilibrium temperature and extend the working time of the battery.

### (5) Embodiment 5

This embodiment provides a capsule endoscope. The other structures of the capsule endoscope are the same as any one of the capsule endoscope structures in Embodiments 1-4, except that, in order to further concentrate heat in the space enclosed by the enclosure of the capsule endoscope, which is conducive to the battery component to increase the working temperature, in the capsule endoscope of this embodiment, a transparent thermal insulation material layer is provided between the image acquisition component and the enclosure 1.

The image acquisition component 4 comprises an illumination unit, a camera, and an image processer. The image acquisition component needs to capture clear images of the human body, so the end portion 1-2 of the enclosure 1 corresponding to the image acquisition component 4 is usually made of transparent material. The image acquisition component 4 emits heat during operation, which is dissipated to the outside through the enclosure 1.

As shown in FIG. 9, a second thermal insulation material layer 10 is disposed between the image acquisition component and the enclosure to reduce heat convection and radiation, concentrating heat inside the enclosure of the capsule endoscope, but without reducing the light transmittance of the end portion 1-2 of the enclosure 1, which would not affect the image capture of the camera. Therefore, a transparent thermal insulation material is chosen to form the second thermal insulation material layer 10.

In one embodiment, a transparent thermal insulation material comprising metal organic ester aerogel is used to the second thermal insulation material layer 10. In one embodiment, the second thermal insulation material layer 10 may be coated on the inner wall of the end portion 1-2 of the enclosure. However, the present invention is not limited to this.

Aerogel materials prepared with metal organic esters have a transparency of 90%, a porosity of 95%, a thermal conductivity of 0.015 W/mK-0.020 W/mK, good thermal insulation properties, and high transparency. They can reduce the heat generated by the image acquisition component from diffusing to the outside of the enclosure, while also taking into account the quality of capturing images.

According to the capsule endoscope of the present embodiment, in addition to timely transferring the heat generated by the image acquisition component to the battery component, it can also reduce the heat generated by the image acquisition component from dissipating to the outside of the enclosure, better concentrating the heat inside the enclosure of the capsule endoscope, allowing the battery to reach a higher thermal equilibrium temperature, and extending the working time of the battery.

In the present invention, the terms “first” and “second” are only used for descriptive purposes and should not be understood as indicating or implying relative importance. The term “multiple” means two or more. For those skilled in the art, the specific meanings of the terms in the present invention may be understood on a case-by-case basis.

The above descriptions are only preferred embodiments of the present invention, and are not intended to limit the present invention. For those skilled in the art, the present invention may have various modifications and changes. Any modifications, equivalent replacements, improvements, etc. made within the spirit and principles of the present invention shall be included within the protection scope of the present invention.

1. A capsule endoscope, comprising:
an enclosure;
a battery component, disposed in an accommodating space enclosed by the enclosure;
a circuit control component, electrically connected to the battery component, and disposed adjacent to the battery component;
an image acquisition component, electrically connected to the circuit control component;
a first thermally conductive element, disposed between the battery component and the circuit control component; and
a second thermally conductive element, in contact with the image acquisition component and the battery component, respectively.

2. The capsule endoscope of claim 1, wherein the first thermally conductive element comprises an insulating and thermally conductive material layer.

3. The capsule endoscope of claim 1, wherein the second thermally conductive element comprises a thermally conductive plate; the thermally conductive plate comprising a first end in contact with the image acquisition component, a second end in contact with the battery component, and an intermediate end in contact with the circuit control component; or the thermally conductive plate having a first end in contact with the image acquisition component and a second end in contact with the battery component.

4. The capsule endoscope of claim 3, wherein the second thermally conductive element comprises a thermally conductive frame and a heat dissipation cavity defined by the thermally conductive frame, and the heat dissipation cavity is filled with a solid-state phase change material.

5. The capsule endoscope of claim 4, wherein at the first end where the second thermally conductive element contacts the image acquisition component, the heat dissipation cavity has a roughened inner surface.

6. The capsule endoscope of claim 3, wherein the second thermally conductive element comprises a thermally conductive frame and a heat dissipation cavity defined by the thermally conductive frame, the heat dissipation cavity is filled with gas-liquid phase change material for heat dissipation, and the inner wall surface of the heat dissipation cavity has a capillary structure.

7. The capsule endoscope of claim 6, wherein at the first end where the second thermally conductive element contacts the image acquisition component, the heat dissipation cavity has an evaporation-promoting structure.

8. The capsule endoscope of claim 1, further comprising a first thermal insulation material layer disposed between the battery component and the enclosure.

9. The capsule endoscope of claim 1, further comprising a second thermal insulation material layer, the image acquisition component is arranged in a space corresponding to one end portion of the enclosure, the second thermal insulation material layer is disposed between the image acquisition component and the enclosure, and the second thermal insulation material layer is a transparent thermal insulation material layer.

10. The capsule endoscope of claim 9, wherein the second thermal insulation material layer is arranged on the inner wall at the end portion of the enclosure, and the transparent thermal insulation material layer comprises metal organic ester aerogel.
